(19) United States
(12) Patent Application Publication
(10) Pub. No.: US 2014/0018303 A1
(43) Pub. Date: Jan. 16, 2014

(54) 3-Amino-2-Hydroxy-4-Phenylbutanoyl-Valyl-Isoleucine, Preparation and Use Thereof

(71) Applicants: Min Rao, Shanghai (CN); Mei Ge, Shanghai (CN); Minyu Luo, Shanghai (CN); Lijun Ruan, Shanghai (CN); Xing Xia, Shanghai (CN); Li Zhu, Shanghai (CN); Lingao Ruan, Shanghai (CN); Huaixia Kang, Shanghai (CN); Tian Yang, Shanghai (CN); Tianjiao Wang, Shanghai (CN); Qiushuang Li, Shanghai (CN); Hanyan Jiang, Shanghai (CN)

(72) Inventors: Min Rao, Shanghai (CN); Mei Ge, Shanghai (CN); Minyu Luo, Shanghai (CN); Lijun Ruan, Shanghai (CN); Xing Xia, Shanghai (CN); Li Zhu, Shanghai (CN); Lingao Ruan, Shanghai (CN); Huaixia Kang, Shanghai (CN); Tian Yang, Shanghai (CN); Tianjiao Wang, Shanghai (CN); Qiushuang Li, Shanghai (CN); Hanyan Jiang, Shanghai (CN)

(73) Assignee: SHANGHAI LAIYI CENTER FOR BIOPHARMACEUTICAL R&D, Shanghai (CN)

(21) Appl. No.: 14/008,195

(22) PCT Filed: Nov. 16, 2011
(86) PCT No.: PCT/CN2011/082278

§ 371 Date: Sep. 27, 2013

(30) Foreign Application Priority Data
Mar. 29, 2011 (CN) 201110076554.6
Mar. 29, 2011 (CN) 201110076555.0

(51) Int. Cl. C07K 5/062

(52) U.S. Cl. CPC C07K 5/06052
USPC 514/19.3; 562/448; 435/71.3

(57) ABSTRACT

The present invention relates to a novel compound, 3-amino-2-hydroxy-4-phenylbutanoyl-valyl-isoleucine with structure showed below.

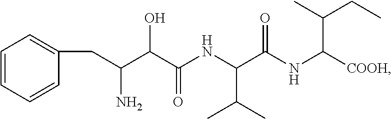 [SMILES: CCC(C)C(NC(=O)C(NC(=O)C(O)C(N)Cc1ccccc1)C(C)C)C(=O)O]

The compound is prepared from fermentation culture Streptomyces parvus CGMCC No. 4027 and is active as inhibitor of aminopeptidase. It can be used for preparation of aminopeptidase inhibitor.

### FIELD OF THE INVENTION

The present invention relates to a novel compound, more particularly to 3-amino-2-hydroxy-4-phenylbutanoyl-valyl-isoleucine, preparation and use thereof.

### BACKGROUND OF THE INVENTION

The inventors obtained a microbial strain, identified as Streptomyces parvus, from a natural soil sample, which was deposited at China General Microbiological Collection Center (CGMCC) on Jul. 20, 2010, with a code of CGMCC No. 4027.

There is little study information with the secondary metabolites from Streptomyces parvus CGMCC No. 4027 except daptomycin.

### DESCRIPTION OF THE INVENTION

A novel compound was isolated and identified from the secondary metabolites of Streptomyces parvus CGMCC No. 4027. Further investigation showed its activity against aminopeptidase.

The object of present invention is to provide a compound 3-amino-2-hydroxy-4-phenylbutanoyl-valyl-isoleucine.

Another object of present invention is to provide a preparation method of the compound 3-amino-2-hydroxy-4-phenylbutanoyl-valyl-isoleucine.

The further object of present invention is to provide the use of the compound 3-amino-2-hydroxy-4-phenylbutanoyl-valyl-isoleucine.

Yet an object of present invention is to provide a pharmaceutical composition as aminopeptidase inhibitor, comprising the compound 3-amino-2-hydroxy-4-phenylbutanoyl-valyl-isoleucine.

The invention relates to a compound of formula (I), named as 3-amino-2-hydroxy-4-phenylbutanoyl-valyl-isoleucine.

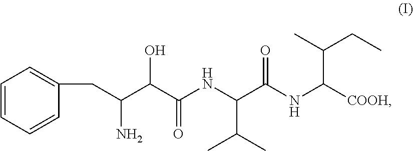 [SMILES: CCC(C)C(NC(=O)C(NC(=O)C(O)C(N)Cc1ccccc1)C(C)C)C(=O)O]

The compound of the invention is prepared from fermentation culture of Streptomyces parvus CGMCC No. 4027.

According to present invention, the compound is active as inhibitor of aminopeptidase, and can be used for prepration of aminopeptidase inhibitor.

According to a preferred embodiment of the invention, the aminopeptidase inhibitor is an inhibitor against aminopeptidase N.

According to present invention, the aminopeptidase inhibitor can be used in preparation of drugs related to cancer treatment, adjuvant treatment, and immunoenhancement.

The pharmaceutical composition as aminopeptidase inhibitor provided in present invention contains the said compound as active ingredient.

Furthermore, the said composition comprises one or more pharmaceutically acceptable vehicles or expicients.

The invention provides a novel compound, and the compound can be used in preparation of drugs as aminopeptidase inhibitor. The function of aminopeptidase N (APN) inhibitor is useful in controlling tumor invasion, metastasis and angiogenesis, as well as in immunoenhancement.

### DESCRIPTION OF THE DRAWINGS

FIG. 1 HPLC profile.

FIG. 2 High resolution MS.

FIG. 3 MS/MS.

FIG. 4 1H-NMR.

FIG. 5 13C-NMR.

FIG. 6 1H-1H COSY.

FIG. 7 HMBC.

### EXAMPLES

The present invention is further described with following examples, which serve to illustrate the invention without limiting the scope thereof.

The microbial strain used in the invention is Streptomyces parvus, which was deposited at China General Microbiological Collection Center (CGMCC) on Jul. 20, 2010, with code of CGMCC No. 4027.

According to present invention, the carbon source of fermentation medium used for Streptomyces parvus CGMCC No. 4027 is selected from 1.0-8.0% of the following: dextrin, soluble starch, citrates, glycerol, glucose, mannitol, rhamnose, L-arabinose, cellobiose, glycogen, salicin, amygdalin, sodium propionate, sodium succinate, sodium acetate, or sodium malate.

The nitrogen source of fermentation is selected from 0.5-5.0% of the following: yeast powder, soybean meal, cottonseed meal, or peanut meal.

According to the invention, said fermentation medium consists of 3.5-4.0% of dextrin, 0.5-1.0% of glucose, 2.0-2.5% of soybean meal, and 0.05-0.15% of ammonium ferrous sulfate. It preferably consists of 3.6% of dextrin, 0.8% of glucose, 2.2% of soybean meal, and 0.1% of ammonium ferrous sulfate.

The formulation (w/v) of the slant culture medium, seed culture medium and fermentation culture medium described in following examples are:
Slant culture: Gause's synthetic agar.
Seed culture: glucose 2%, glycerol 2%, soluble starch 6%, soybean meal 5%, KH₂PO₄ 0.02%, MgSO₄.7H₂O 0.04%, initial pH 7.3-7.5.
Fermentation culture: soybean meal 2.2%, dextrin 3.6%, glucose 0.8%, Fe(NH₄)₂(SO₄)₂ 6H₂O 0.1%,initial pH 7.3-7.5.

### Example 1

### Preparation of the Compound

### 1.1. Fermentation

Transfer the stock culture of strain CGMCC No. 4027 onto slant culture medium, and incubate at 28° C.-30° C. for 4 days. Transfer the slant culture into shaking flasks containing seed culture medium which have been sterilized at 121° C. for 30 min. Cultivate the seed at 28-30° C. on a rotary shaker at 200-230 rpm for 46-50 h. Transfer the seed culture into a 5 L jar fermentor containing sterile fermentation medium. The inoculation volume is 5%. Carry out the fermentation in the jar fermentor with agitation at 400-500 rpm, temperature at 28-30° C., and aeration at 7-9 L/min, for a total period of 4-6 days.

### 1.2. Isolation and Purification

The broth is harvested and placed at 4° C. overnight. Remove the precipitate by centrifugation at 4000 rpm, 4° C., for 30 min. The supernatant is subject to macroporous resin, which is selected from: XAD-1600, SP850, or JD-1, by either static or dynamic mode. The resin is then eluted with double volume of purified water, 10%, 30%, 50%, 75%, and 100% of ethanol. Collect the fraction of the 50% elute and concentrate it under reduced pressure at 40° C. Dilute the concentrate by 10 fold with 30% acetonitrile and centrifuge at 12000 rpm for 10 min. Load 15-25 ml of the supernatant to a column for preparative chromatography (Biotage Si 25+M 1603-2, USA) at room temperature. Elute the column with 30% acetonitrile at flow rate of 3-5 ml/min, and collect the fraction of 200-400 ml. The collected elute is then concentrate under reduced pressure at 40° C., and the concentrate is subject to preparative HPLC by DIONEXP680 system or Agilent 1100 DAD system. The fraction containing target compound is concentrated and dried under reduced pressure at 40° C., and a purified product is obtained. The compound is pale yellow in color, soluble in methanol, ethanol, water and acetonitrile; insoluble in chloroform.
Conditions set for preparative HPLC:
Column—YMC CombiPreP ODS-A, 5 μm, 20×250 mm
Temperature—Room temperature
Detector—DAD (210 nm, 223 nm, 254 nm, 280 nm)
Mobile phase (v/v)—25% acetonitrile & 75% water with 0.1% TFA
Flow rate: 8 ml/min
Loading volume: 60-700 μl
Isocratic elution

### Example 2

### Identification of the Compound

Apply the purified sample from preparative HPLC to a Agilent 1100 DAD system for analysis using following conditions:
Column—Feiluomen C18 column, 5 μm, 4.6×250 mm
Temperature—30° C.
Detector—VWD 210 nm
Mobile phase (v/v)—25% acetonitrile & 75% water with 0.1% TFA
Flow rate: 1 ml/min
Loading volume: 5 μl
Isocratic elution

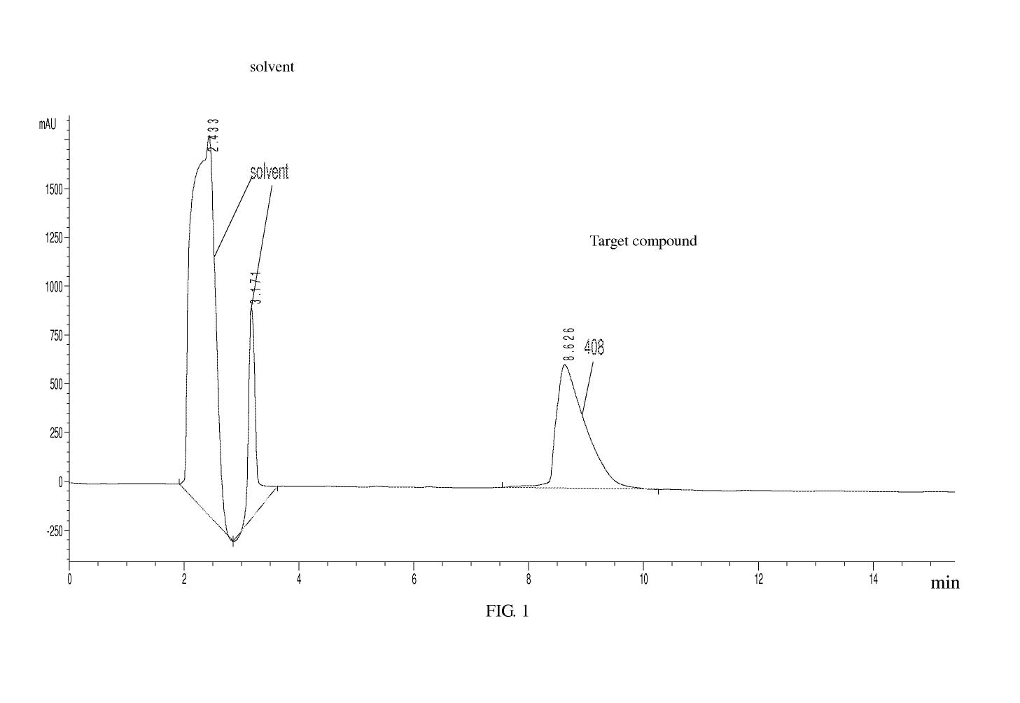

FIG. 1 shows the target peak is around 8.6 min. When the two peaks representing solvent are removed, the final purity is above 95%.

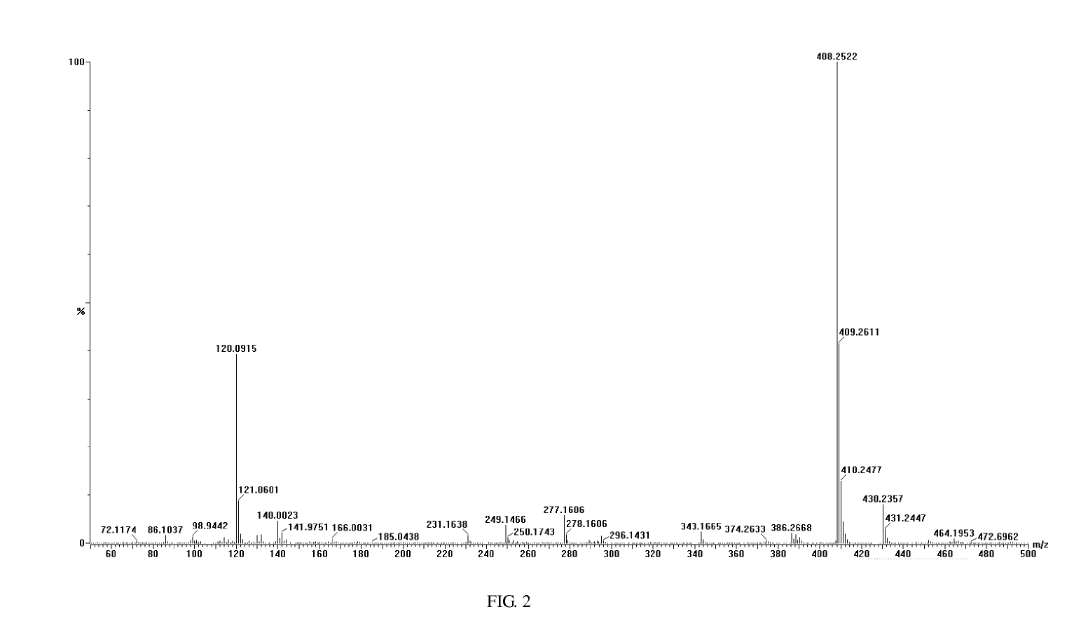

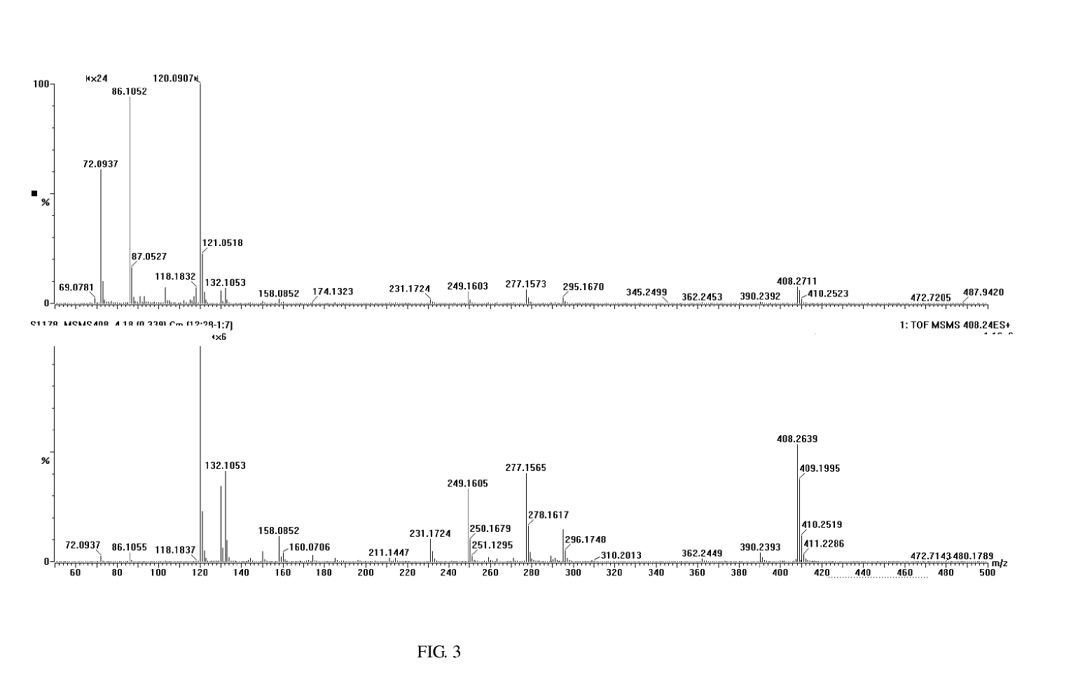

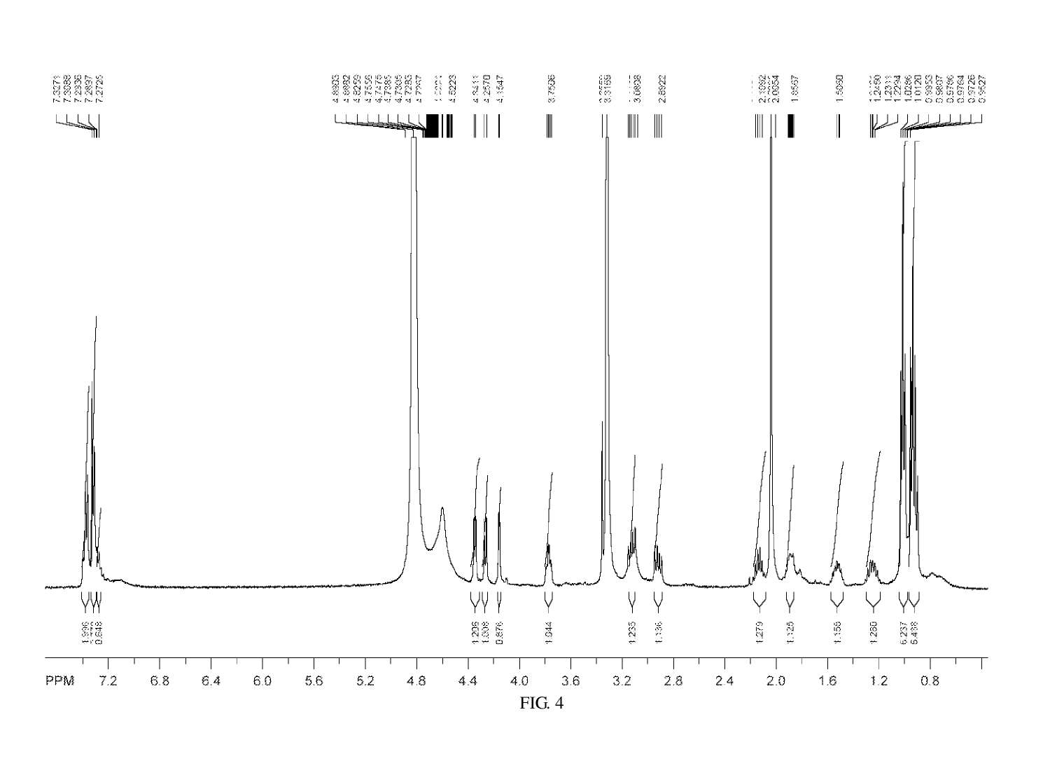

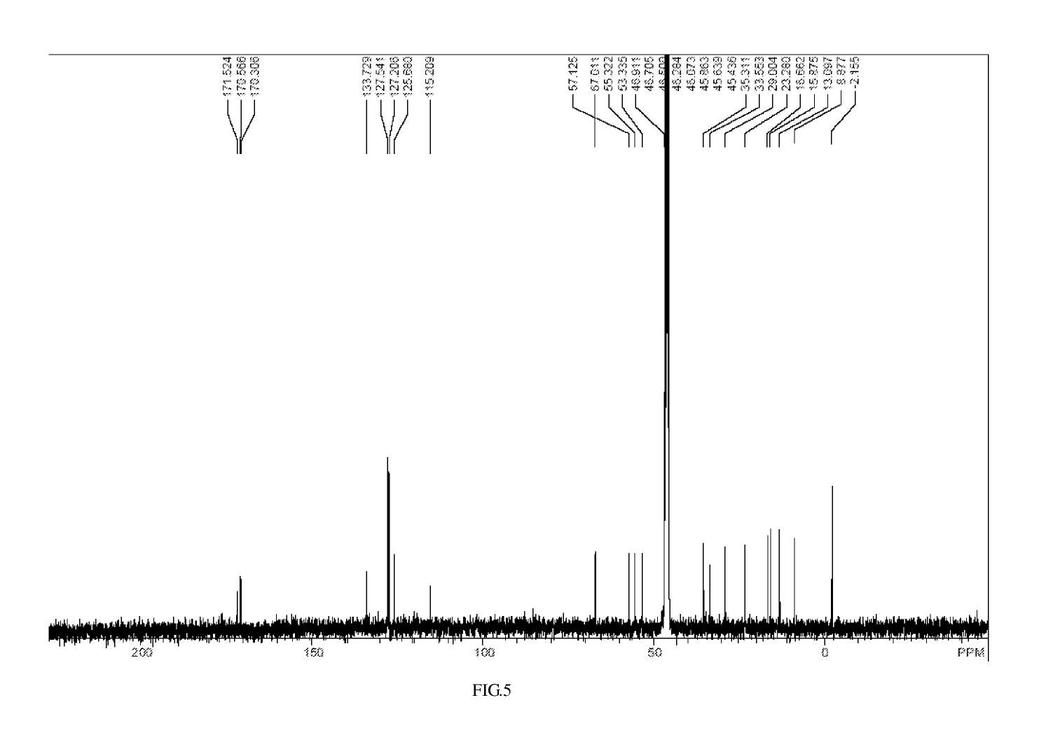

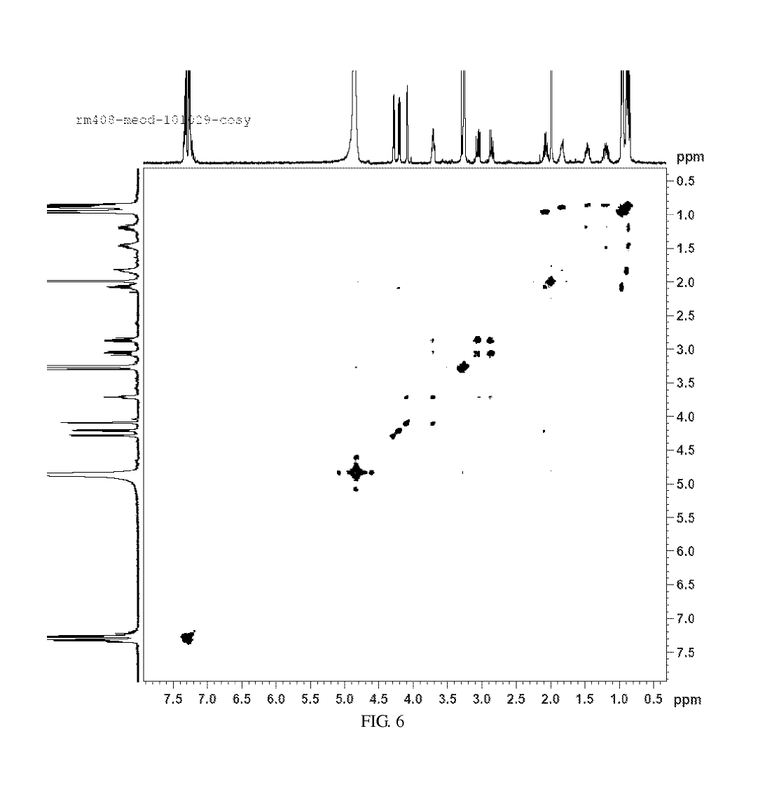

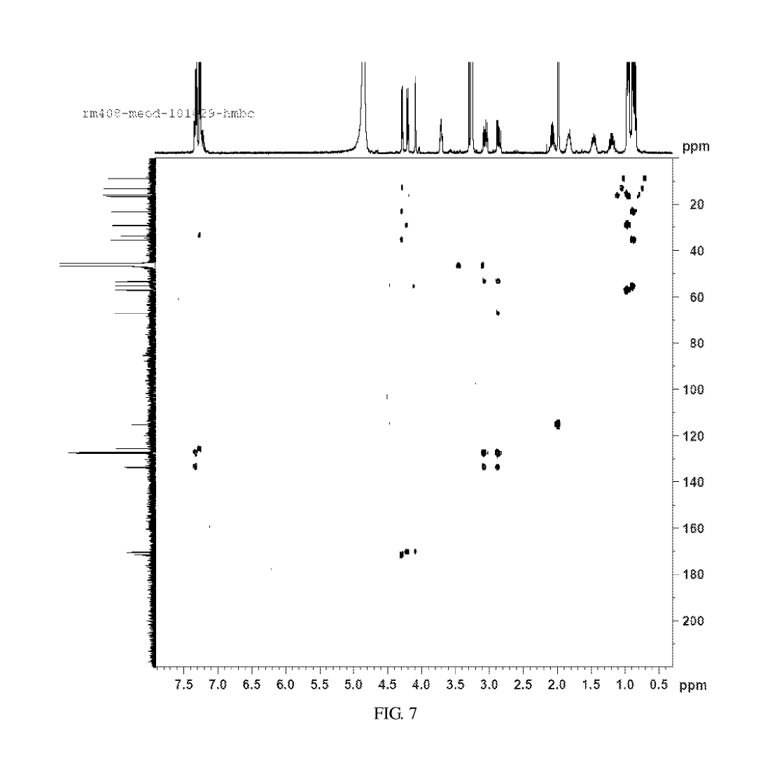

According to high resolution MS analysis (FIG. 2) the molecular weight of the compound is 407.2522, molecular formula C₂₁H₃₃N₃O₅. MS-MS analysis (FIG. 3), ¹H-NMR (FIG. 4), ¹³C-NMR (FIG. 5), ¹H-¹H COSY (FIG. 6) and HMBC (FIG. 7) are performed further. Table 1 shows the NMR data.

TABLE 1
NMR Data

| Moiety | ¹HNMR | | ¹³CNMR | HMBC |
|---|---|---|---|---|
| Ile | qc | | 174.4 | |
| | 2-CH | 4.29(d, J = 6.0) | 58.2 | Qc, C3, C4, C6 |
| | 3-CH | 1.84(m) | 38.2 | |
| | 4-CH2 | 1.47(m) | 26.2 | |
| | | 1.21(m) | | |
| | 5-CH3 | 0.87(dd, J = 6.8, 14.4) | 16.0 | C2, C3, C4 |
| | 6-CH3 | 0.84(dd, J = 6.8, 14.4) | 11.8 | C2, C3, C4 |
| Val | qc | | 173.5 | |
| | 2′-CH | 4.19(d, J = 8.0) | 60.1 | Qc, C3′ |
| | 3′-CH | 2.10(dd, J = 5.2, 13.2) | 31.9 | |
| | 4′-CH3 | 0.97(t, J = 6.8) | 19.6 | C2′, C3′ |
| | 5′-CH3 | 0.95(t, J = 6.8) | 18.8 | C2′, C3′ |
| AHPA | qc | | 173.2 | |
| | 2″-CH | 4.09(d, J = 3.6) | 69.9 | Qc |
| | 3″-CH | 3.72(m) | 56.3 | |
| | 4″-CH2 | 3.07(dd, J = 7.8, 13.5) | 36.5 | C2″, C3″, |
| | | 2.89(dd, J = 7.8, 13.5) | | C4″a-qc |
| | 4″a-qc | | 136.6 | |
| | 4″b-CH | 7.25(m) | 130.5 | |
| | 4″c-CH | 7.35(m) | 130.1 | |
| | 4″d-CH | 7.24(m) | 128.6 | |
| | 4″e-CH | 7.35(m) | 130.1 | |
| | 4″f-CH | 7.25(m) | 130.5 | |

The chemical structure of the compound was identified as the following, and there has been no same structure ever reported.

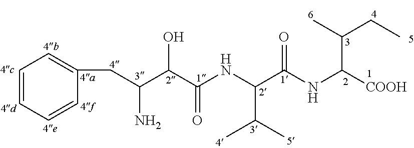 [SMILES: CCC(C)C(NC(=O)C(NC(=O)C(O)C(N)Cc1ccccc1)C(C)C)C(=O)O]

### Example 3

### Bioassay of the Compound

Methodology as reported in “J. Microbiol. Biotechnol., 1995, 5(1):36-40” is followed to analyze the bioactivity of 3-amino-2-hydroxy-4-phenylbutanoyl-valyl-isoleucine, taking bestatin, a typical APN inhibitor used in leukemia treatment, as positive control.

The procedure for the assay is as follows: apply 100 μl (1 mg/ml in a 0.1M Tris-HCl, pH 7.0) of substrate (L-leucine-p-nitroanilide) to a 96-well plate, mix with 20 82 l (0.1M Tris-HCl, pH 7.0) of the compound to be assayed of different concentration (0.86, 1.71, 2.57, 4.29, 8.57 μg/ml). Incubate at 37° C. for 3 min, followed by addition of 20 μl (2 mU, 0.1M Tris-HCl, pH 7.0) aminopeptidase N (L5006-25UN, Sigma), incubate at 37° C. for 30 min. Read the absorbance at 405 nm.

The inhibition rate is presented as:

Inhibition rate=(A−B)/A*100%

wherein:
A is the absorbance tested without inhibitor in the system.
B is the absorbance detected in the system containing different concentration of the compound. Use 0.1M Tris-HCl (pH 7.0) to normalize the buffer. Table 2 shows the inhibition rate (IR) of the compound of the invention at five different concentrations against APN.

TABLE 2
Inhibition Rate against APN

| | Concentration (μg/ml) | | | | |
|---|---|---|---|---|---|
| | 0.86 | 1.71 | 2.57 | 4.29 | 8.57 |
| IR of positive control (%) | 41.61 | 44.37 | 47.13 | 52.65 | 66.45 |
| IR of the compound (%) | 42.02 | 51.40 | 65.14 | 70.79 | 77.91 |

Result from Table 2 indicated that the compound, 3-amino-2-hydroxy-4-phenylbutanoyl-valyl-isoleucine, isolated form the secondary metabolites of Streptomyces parvus CGMCC No.4027, is active as an inhibitor against APN, and its inhibition is stronger than bestatin at the same concentration.

APN is a kind of membrane-binding exopeptidase containing zinc which is expressed abundantly on the surface of tumor cells. It is closed related to the growth of primary/secondary tumor and angiogenesis. It even facilitates tumor proliferation and differentiation. It works to degrade the major components of extracellular matrix (ECM), and enhances tumor cell growth and metastasis. In addition, it is expressed on the surface of antigen-presenting cells degrading various immune-active substances. This results in immunity decline, weakening of the activity of macrophages and NK cells against tumor cells.

Therefore tumor invasion, metastasis and angiogenesis can be intervened by inhibiting APN activity. Meanwhile the chemo-taxis of granulocyte is enhanced and immunity intensified. As APN has become a significant target of anti-tumor and anti-virus medication, APN inhibitor with high efficiency, low toxicity and high selectivity is hopeful to be applied in cancer treatment.

Bestatin is a compound with APN inhibiting activity found in the culture of Streptomyces olivoreticuli. It was marketed in 1987 as anti-tumor drug which showed significant effect against acute leukemia and malignant melanoma.

According to the result of the example, 3-amino-2-hydroxy-4-phenylbutanoyl-valyl-isoleucine showed higher inhibitory activity compared to bastatin. It can be used in preparation of drugs related to cancer treatment, adjuvant treatment and immunoenhancement.

Moreover, for those skilled in the art, these drugs contain not only effective amount of 3-amino-2-hydroxy-4-phenylbutanoyl-valyl-isoleucine, but also pharmaceutically acceptable vehicles or expicients, such as solvant, diluent, etc. 3-amino-2-hydroxy-4-phenylbutanoyl-valyl-isoleucine mixed with one or more pharmaceutically acceptable vehicles or expicients forms a drug formulation, and it is used in cancer treatment, adjuvant treatment, and immunoenhancement.

1-10. (canceled)

11. A compound having the following formula:

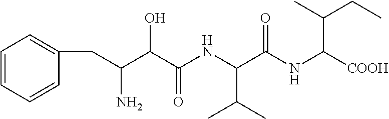 [SMILES: CCC(C)C(NC(=O)C(NC(=O)C(O)C(N)Cc1ccccc1)C(C)C)C(=O)O]

12. A method of preparing the compound of claim 11 comprising:
a) transferring a stock culture of Streptomyces parvus CGMCC No. 4027 into a slant culture medium and incubating;
b) transferred the slant culture into a sterilized seed culture medium and cultivating;
c) fermenting the seed culture in a fermentation medium comprising a carbon source and a nitrogen source;
d) isolating and purifying a fraction from the fermentation medium containing a target compound; and
e) identifying the compound of claim 11 from the fraction.

13. The method of claim 12, wherein said slant culture medium comprises Gaise's synthetic agar.

14. The method of claim 12, wherein said seed culture comprises a combination of glucose, glycerol, soluble starch, soybean meal, KH₂PO₄, and MgSO₄ with an initial pH of about 7.3-7.5.

15. The method of claim 12, wherein said fermentation medium comprises soybean meal, dextrin, glucose, Fe(NH₄)₂(SO₄)₂ with an initial pH of about 7.3-7.5.

16. The method of claim 12, wherein said isolation and purification step comprises chromatography or HPLC for eluting the fraction containing target compound.

17. The method of claim 16, wherein said eluted fraction target compound is further concentrated and dried for obtaining the purified compound.

18. The method of claim 12, wherein said compound of claim 11 is identified by high resolution MS analysis.

19. The method of claim 12, wherein said compound is an aminopeptidase inhibitor.

20. The method of claim 19, wherein said aminopeptidase inhibitor is an aminopeptidase N inhibitor.

21. A method for a cancer treatment, a cancer adjuvant treatment, or an immunoenhancement comprising administering to a subject in need an effective amount of the compound of claim 11.

22. A composition for a cancer treatment, a cancer adjuvant treatment, or an immunoenhancement comprising an effective amount of compound 11 as an aminopeptidase inhibitor.

23. The composition of claim 22, wherein said aminopeptidase inhibitor is an aminopeptidase N inhibitor.

24. The composition of claim 22, wherein said composition further comprises one or more pharmaceutically acceptable vehicles or expicients.
